(19) United States
(12) Patent Application Publication
(10) Pub. No.: US 2016/0329504 A1
(43) Pub. Date: Nov. 10, 2016

(54) AROMATIC DERIVATIVES HAVING ELECTRON DONATING GROUP AND ELECTRON ACCEPTING GROUP AND ORGANIC LIGHT EMITTING DIODE USING THE SAME

(71) Applicant: National Tsing Hua University, Hsinchu (TW)

(72) Inventors: Chien-Hong CHENG, Hsinchu (TW); Chih-Chun LIN, Hsinchu (TW); Yi-Ching WU, Hsinchu (TW)

(21) Appl. No.: 14/850,643

(22) Filed: Sep. 10, 2015

(30) Foreign Application Priority Data
May 5, 2015 (TW) 104114331

(51) Int. Cl. H01L 51/00; C09K 11/06; C07C 255/58; C07D 209/88; C07C 317/32; C07D 209/86; C09K 11/02

(52) U.S. Cl. CPC H01L 51/0072; C07D 209/86; C09K 11/06; C09K 11/025; C07D 209/88; C07C 317/32; H01L 51/0059; H01L 51/006; C07C 255/58; H01L 51/5012

(57) ABSTRACT

Aromatic derivatives having an electron donating group and an electron accepting group at each end are provided. The aromatic derivatives of the present invention can emit blue or green light and may function as a host material or a dopant material. An OLED device using the aromatic derivatives is also herein disclosed.

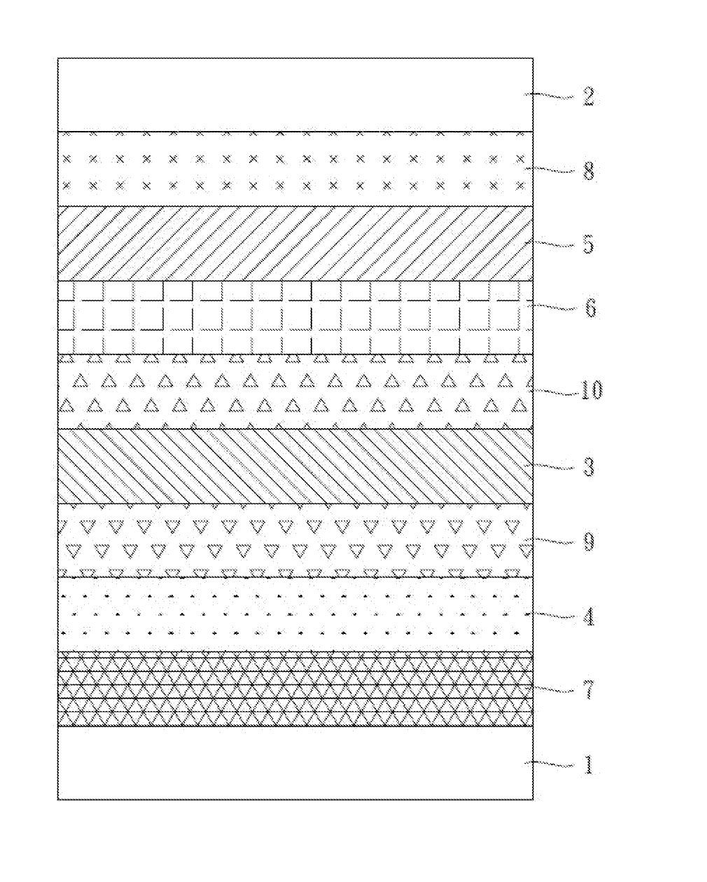

### BACKGROUND OF THE INVENTION

1.Field of the Invention

The present invention relates to an aromatic derivative and an OLED using the same, particularly to an aromatic derivative whose two ends respectively have an electron donating group and an electron accepting group and an OLED using the same.

2.Description of the Prior Art

The organic light emitting diode (OLED), also called the organic electroluminescent device, is a light emitting diode (LED) using an organic layer as the active layer. As OLED features self-luminescence, wide viewing angle (>170°), fast response (˜μs), high contrast, high efficiency, low power consumption, high brightness, low operating voltage (3-10V), thinner thickness (<2 mm) and flexibility, it has been gradually used in flat panel display devices for the last few years. Distinct from liquid crystal display (LED) devices, the OLED display device has a self-luminescent OLED pixel array. Therefore, the OLED display device is exempted from using a backlight module. In order to apply OLEDs to full color display devices, the manufacturers have to develop red light, green light and blue light OLEDs having high light emitting efficiency.

The excitons generated by the recombination of electrons and holes may have a triplet state or a singlet state in their spin. The singlet exciton generates fluorescence, and the triplet exciton generates phosphorescence. The light emitting efficiency of phosphorescence is 3 times higher than that of fluorescence. Introducing heavy metals into the light emitting structure will cause intense spin-orbit coupling and mix the triplet excitons and singlet excitons, whereby the internal quantum efficiency (IQE) is greatly increased as high as 100%. Therefore, manufacturers have adopted phosphorescent metal complexes as the phosphorescent dopants in the light emitting layer of OLED in recent years. Normally, a doping process is used to dope a light emitting material into a host material to inhibit the self-quenching phenomenon. The host material is a critical subject in developing OLED devices. The host material must have characteristics of superior carrier capture ability, superior energy conversion ability, high glass transition temperature, superior thermal stability, energy gaps suitable for triplet state and singlet state. However, the conventional host materials are hard to completely meet the abovementioned conditions. Therefore, host materials still have much room to improve.

As blue light OLED is a critical element to realize OLED-based lighting devices and display devices, developing efficient blue light OLED is an important task for researchers and manufacturers. So far, many research teams have successfully fabricated efficient blue light fluorophores and OLEDs using the same. However, the blue light OLED materials whose CIEy (Commission Internationale d'Énclairage y coordinate value) ≦0.15 are still very rare at present. For the time being, the industry still lacks superior OLED compounds meeting the abovementioned requirements.

Therefore, a novel and efficient blue light OLED material is the target the manufacturers and researchers are eager to achieve.

### SUMMARY OF THE INVENTION

One objective of the present invention is to provide a novel aromatic derivative whose two ends respectively have an electron donating group and an electron accepting group.

In one embodiment, the aromatic derivative of the present invention is expressed by Representative Formula (I):

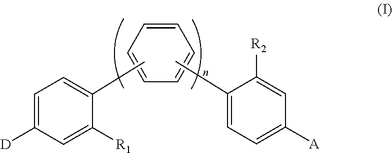

wherein n is an integer ranging from 0-3; each of R₁ and R₂ is a independently hydrogen atom, a C1-C6 alkyl group, or a phenyl group; A is a substituted or unsubstituted benzenesulfonic acid, or a substituted or unsubstituted benzene dinitrile group; D is a substituted or unsubstituted diarylamine group, or a substituted or unsubstituted nitrogen-containing heteroaryl group.

Another objective of the present invention is to provide an organic light emitting diode (OLED) having high efficiency.

In one embodiment, the OLED of the present invention comprises a cathode, an anode and a light emitting layer interposed between the cathode and the anode, wherein the light emitting layer includes the abovementioned aromatic derivative as the light emitting material.

The aromatic derivative of the present invention can emit blue light or green light and function as a host light emitting material or a dopant light emitting material.

Below, embodiments are described in detail in cooperation with the attached drawings to make easily understood the objectives, technical contents, characteristics and accomplishments of the present invention.

### BRIEF DESCRIPTION OF THE DRAWINGS

The sole FIGURE is a diagram schematically showing the structure of a light emitting element using aromatic derivatives according to one embodiment of the present invention.

### DESCRIPTION OF THE PREFERRED EMBODIMENT

The present invention provides a blue-light aromatic fluorescent material expressed by Representative Formula (I):

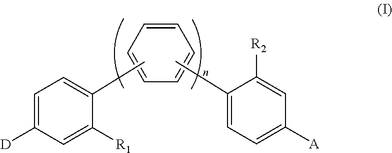

wherein n is an integer ranging from 0-3, preferably 0 or 1; each of R₁ and R₂ is independently a hydrogen atom, a C1-C6 alkyl group, or a phenyl group, preferably a hydrogen atom or a methyl group; the connection positions may be the para-, meta-, or ortho-positions of the benzene rings.

Group D is an electron donating group. Group D is a substituted or unsubstituted diarylamine group, or a substituted or unsubstituted nitrogen-containing heteroaryl group. The nitrogen-containing heteroaryl group may be but is not limited be pyrrolyl, oxazolyl, imidazolyl, thiazolyl, pyridyl, pyrimidinyl, quinazolinyl, quinolyl, isoquinolyl, or indolyl.

In one embodiment, Group D is bonded to the benzene ring through a nitrogen atom. The nitrogen-containing heteroaryl group may be a heteroaryl group with two benzene rings fused with a central heteroaryl group, which may be but is not limited to be carbazole, 4aH-phenoxazine, or acridine. The nitrogen-containing heteroaryl group include but are not limited to be the groups shown below:

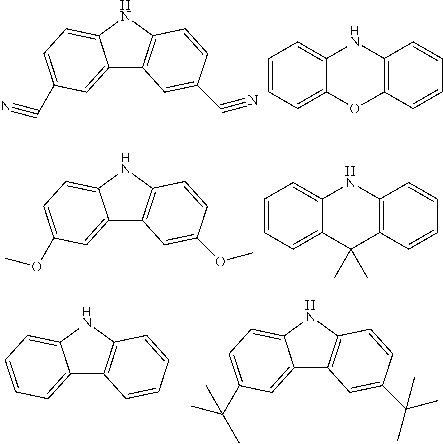

In one embodiment, the diarylamine group is selected from a group consisting of:

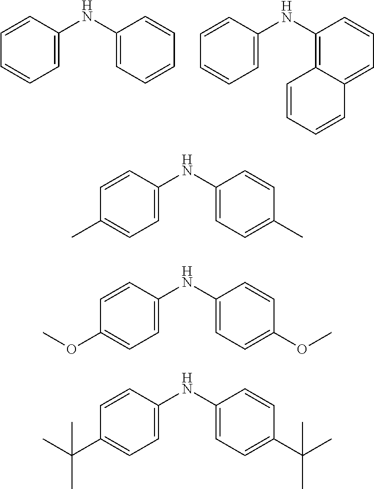

Group A is an electron accepting group. Group A is a substituted or unsubstituted benzenesulfonic acid, or a substituted or unsubstituted benzene dinitrile group, where the dinitrile group is 3, 5-benzene dinitrile. In one embodiment, Group A is selected from a group consisting of:

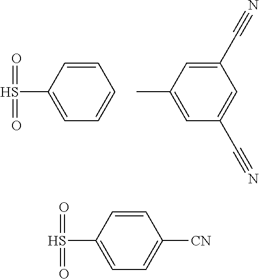

The substitute group of group A or group D is selected from a group consisting of halogen atoms, aryl groups, alkenyl groups, C1-C20 alkyl groups, alkynyl groups, the cyano group (CN), the trifluoromethyl group (CF₃), alkylamino groups, amino group, alkoxy groups, heteroaryl groups, halogen substituted aryl groups, halogen substituted aralkyl groups, haloalky substituted aryl groups, haloalkyl substituted aralkyl groups, aryl substituted C1-C20 alkyl groups, cycloalkyl groups, C1-C20 alkoxy group, C1-C20 alkyl substituted amino groups, haloalkyl substituted amino groups, aryl substituted amino groups, heteroaryl substituted amino groups, aryl substituted phosphine oxide groups, C1-C20 alkyl substituted phosphine oxide groups, haloalkyl substituted phosphine oxide groups, halogen substituted phosphine oxide groups, the nitro group, the carbonyl group, aryl substituted carbonyl groups, heteroaryl substituted carbonyl groups, and halogen substituted C1-C20 alkyl groups.

In the specification, aryl is referred to a hydrocarbon group having one or more aromatic rings. The aryl group may be but is not limited to be a phenyl group, a phenylene group, a naphthyl group, a naphthylene group, a pyrenyl group, an anthryl group, and a phenanthryl group. The heteroaryl is referred to a hydrocarbon group having one or more aromatic rings, and the aromatic ring has at least one hetero atom (such as nitrogen, oxygen or sulfur). The heteroaryl group may be but is not limited to a furyl group, a furylene group, a fluorenyl group, a pyrrolyl group, a thienyl group, a oxazolyl group, an imidazolyl group, a thiazolyl group, a pyridyl group, a pyrimidinyl group, a quinazolinyl group, a quinolyl group, an isoquinolyl group, and an indolyl group.

It should be particularly mentioned: each of the alkyl groups, the alkenyl groups, the alkynyl groups, the cycloalkyl groups, cycloalkenyl groups, the heteroaryl groups, the heterocycloalkenyl groups, the aryl groups and the heteroaryl groups may be a substituted or unsubstituted group.

The substitute groups of the cycloalkyl groups, cycloalkenyl groups, the heterocycloalkyl groups, the heteroaryl groups, the aryl groups and the heteroaryl groups include but are not limited to be C1-C10 alkyl groups, C2-C10 alkenyl groups, C2-C10 alkynyl groups, C3-C20 cycloalkyl groups, C3-C20 cycloalkenyl groups, C1-C20 heterocycloalkyl groups, C1-C20 heterocycloalkenyl groups, C1-C10 alkoxy groups, aryl groups, aryloxy groups, heteroaryl groups, heteroaryloxy groups, amino group, C1-C10 alkylamino groups, C1-C20 dialkylamino groups, arylamine groups, diarylamine groups, C1-C10 alkylsulfonamino groups, arylsulfonamino groups, C1-C10 alkylimino groups, arylimino groups, C1-C10 alkylsulfonimino groups, arylsulfonimino groups, hydroxyl group, halogens, thio groups, C1-C10 alkylthio groups, arylthio groups, C1-C10 alkylsulfonyl groups, arylsulfonyl groups, acylamino groups, aminoacyl groups, aminothioacyl groups, amido groups, amidino groups, guanidine groups, ureido groups, thioureido groups, a nitrile group, a nitro group, a nitrosyl group, an azido group, an acyl group, a sulfuryl group, an acyloxy group, a carboxyl group, and carboxylic acid esters. The substitute groups of the alkyl groups, the alkenyl groups, the alkynyl groups may be all the abovementioned groups except the C1-C10 alkyl groups. Besides, the cycloalkyl groups, cycloalkenyl groups, the heterocycloalkyl groups, the heterocycloalkenyl groups, the aryl groups and the heteroaryl groups can be fused to each other.

Refer to General Synthesis Formula (1) of the compound of the present invention:

General Synthesis Formula (1)

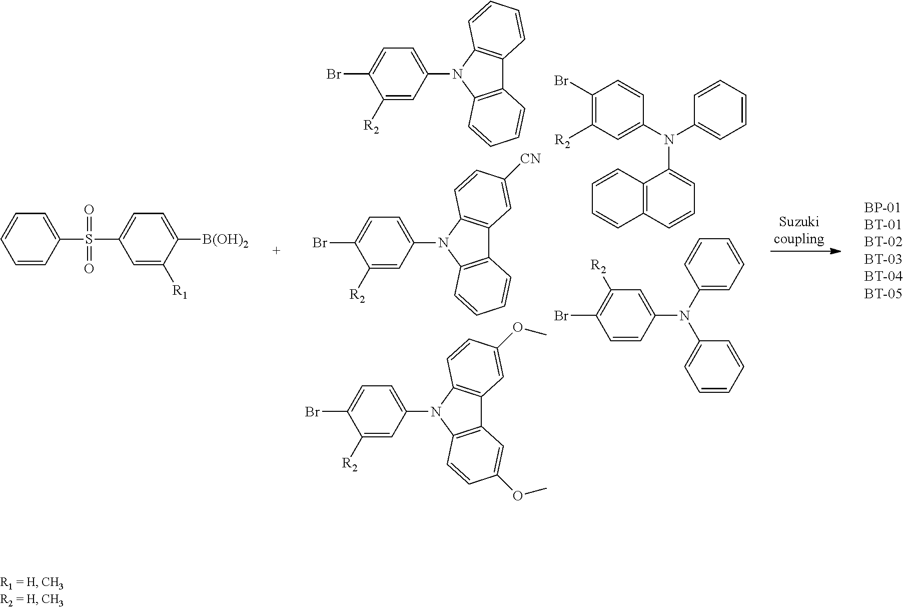

wherein group A in Representative Formula (1) is exemplified by the benzenesulfonic acid radical; group D is exemplified by carbazole or diarylamine. It should be noted: the initial reactants having a given number of benzene rings will output an aromatic derivative having a corresponding number of benzene rings.

In the synthesis reaction, take a derivative of (4-(phenylsulfonyl)phenyl)boronic acid (1.1 mmole) and a derivative containing an amine group (1 mmole) into a high pressure tube; add toluene (3 mL), ethanol (1 mL), 2M potassium carbonate aqueous solution (1 mL), and Pd(PPh₃)₄(0.1 mmole) into the high-pressure tube; The system was vacuumed and refilled with nitrogen three times, and then seal the tube; place the tube in an oil bath at a temperature of 80° C. to undertake a Suzuki coupling reaction for 8 hours, and then let the tube cool down to the ambient temperature; use diatomite to filter the resultant solution to remove metal; use ethyl acetate to extract the organic layer and remove water (the solvent); use a column chromatography device to purify the resultant; use a high temperature sublimation process to further purify the resultant to obtain the product of the synthesis reaction.

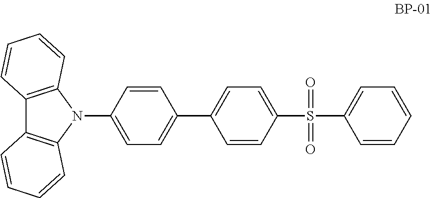 [SMILES: O=S(=O)(c1ccccc1)c1ccc(-c2ccc(-n3c4ccccc4c4ccccc43)cc2)cc1]

The Suzuki coupling reaction of (4-(phenylsulfonyl)phenyl)boronic acid and 9-(4-bromophenyl)-9H-carbazole generates Product BP-01.

¹H NMR (400 MHz, CDCl₃), δ(ppm): 8.15-8.17(m, 2H), 8.07-8.04(m, 2H) 8.01-7.99(m, 2H) 7.80-7.76(m, 4H) 7.68-7.65 (m, 2H) 7.59-7.57 (m, 1H) 7.56-7.51 (m, 2H) 7.46-7.44 (m, 2H) 7.43-7.39 (m, 2H) 7.31-7.27 (m, 2H)

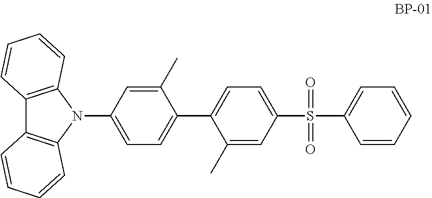 [SMILES: Cc1cc(-n2c3ccccc3c3ccccc32)ccc1-c1ccc(S(=O)(=O)c2ccccc2)cc1C]

The Suzuki coupling reaction of (2-methyl-4-(phenylsulfonyl)phenyl)boronic acid and 9-(4-bromo-3-methylphenyl)-9H-carbazole generates Product BT-02.

¹H NMR (400 MHz, CDCl₃), δ(ppm): 8.14 (d, J=8.0, 2H), 8.04-8.01(m, 2H) 7.89(s, 1H) 7.86-7.83(dd, J=8.0, 1.6, 1H) 7.61-7.53 (m, 3H) 7.46 (d, J=8.0, 3H) 7.44-7.39 (m, 3H) 7.35 (d, J=8.0, 1H) 7.30-7.27 (m, 2H) 7.22 (d, J=8.0, 1H) 2.21 (s, 3H) 2.09 (s, 3H)

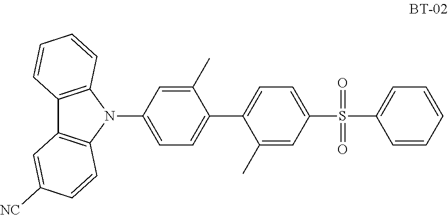 [SMILES: Cc1cc(-n2c3ccccc3c3cc(C#N)ccc32)ccc1-c1ccc(S(=O)(=O)c2ccccc2)cc1C]

The Suzuki coupling reaction of (2-methyl-4-(phenylsulfonyl)phenyl)boronic acid and 9-(4-bromo-3-methylphenyl)-3,6-dimethoxy-9H-carbazole generates Product BT-02.

¹H NMR (400 MHz, CDCl₃), δ(ppm): 8.44 (s, 1H), 8.15 (d, J=8.0, 1H) 8.04-8.01(m, 2H) 7.91-7.90 (m, 1H) 7.87-7.85 (dd, J=8.0, 0.8, 1H) 7.66-7.63 (m, 1H) 7.61-7.59 (m, 1H) 7.58 (d, J=8.0, 1H) 7.56-7.52 (m, 1H) 7.51-7.43 (m, 4H) 7.40-7.37(m, 2H) 7.34 (d, J=8.0, 1H) 7.26 (d, J=8.0, 1H) 2.21 (s, 3H) 2.11 (s, 3H)

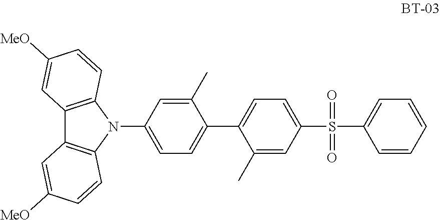 [SMILES: COc1ccc2c(c1)c1cc(OC)ccc1n2-c1ccc(-c2ccc(S(=O)(=O)c3ccccc3)cc2C)c(C)c1]

The Suzuki coupling reaction of (2-methyl-4-(phenylsulfonyl)phenyl)boronic acid and 9-(4-bromo-3-methylphenyl)-9H-carbazole-3-carbonitrile generates Product BT-03.

¹H NMR (400 MHz, CDCl₃), δ(ppm): 8.03-8.00(m, 2H) 7.88(s, 1H) 7.84-7.82 (m, 1H) 7.62-7.56 (m, 2H) 7.55-7.52 (m, 3H) 7.44-7.43 (m, 1H) 7.40-7.34 (m, 3H) 7.33 (d, J=8.0, 1H) 7.19 (d, J=8.0, 1H) 7.05 (d, J=4.0, 1H) 3.94(s, 6H) 2.20 (s, 3H) 2.08 (s, 3H)

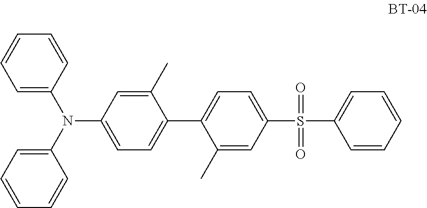 [SMILES: Cc1cc(N(c2ccccc2)c2ccccc2)ccc1-c1ccc(S(=O)(=O)c2ccccc2)cc1C]

The Suzuki coupling reaction of (2-methyl-4-(phenylsulfonyl)phenyl)boronic acid and 4-bromo-3-methyl-N,N-diphenylaniline generates Product BT-04.

¹H NMR (400 MHz, CDCl₃), δ(ppm): 7.99-7.97(m, 2H) 7.81(s, 1H) 7.77-7.74 (m, 1H) 7.57-7.50 (m, 3H) 7.27-7.23 (m, 3H) 7.11-7.09 (m, 4H) 7.03-6.99 (m, 3H) 6.95-6.94 (m, 1H) 6.90-6.87 (m, 1H) 6.83 (d, J=8.0, 1H) 2.14 (s, 3H) 1.87 (s, 3H)

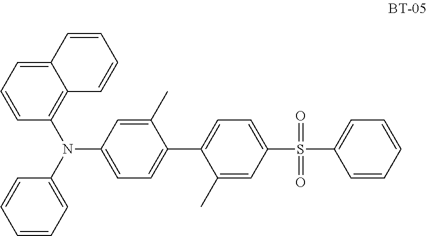 [SMILES: Cc1cc(N(c2ccccc2)c2cccc3ccccc23)ccc1-c1ccc(S(=O)(=O)c2ccccc2)cc1C]

The Suzuki coupling reaction of (2-methyl-4-(phenylsulfonyl)phenyl)boronic acid and N-(4-bromo-3-methylphenyl)-N-phenylnaphthalen-l-amine generates Product BT-05.

¹H NMR (400 MHz, CDCl₃), δ(ppm): 7.98-7.93(m, 3H) 7.88-7.86(d, J=8.0, 1H) 7.79-7.72 (m, 2H) 7.58-7.43 (m, 6H) 7.37-7.34 (m, 2H) 7.22-7.17 (m, 3H) 7.04 (d, J=8.0, 1H) 6.94-6.90 (m, 2H) 6.83-6.81 (m, 2H) 6.78(d, J=8.0, 1H) 2.11 (s, 3H) 1.83 (s, 3H)

Refer to General Synthesis Formula (2) of the compound of the present invention:

General Synthesis Formula (2)

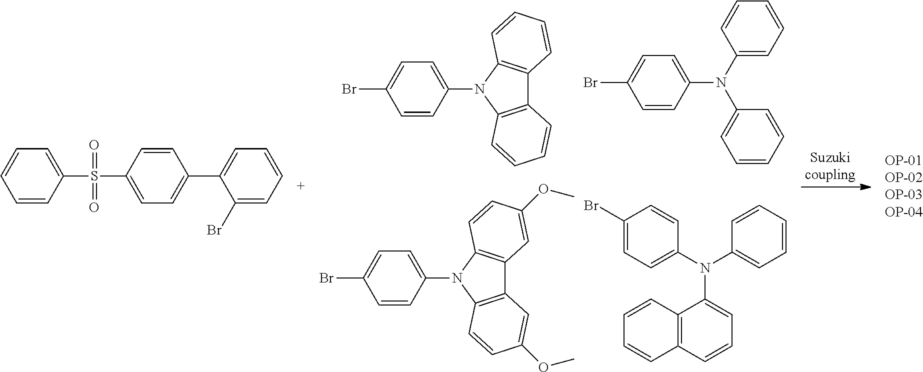

wherein group A in Representative Formula (2) is exemplified by the benzenesulfonic acid radical; group D is exemplified by carbazole or diarylamine. It should be noted: the initial reactants having a given number of benzene rings will output an aromatic derivative having a corresponding number of benzene rings.

In the synthesis reaction, take 2-bromo-4′-(phenylsulfonyl)-1,1′-biphenyl (1.1 mmole) and a derivative containing an amine group (1 mmole) into a high pressure tube; add toluene (3 mL), ethanol (1 mL), 2M potassium carbonate aqueous solution (1 mL), and Pd(PPh₃)₄ (0.03 mmole) into the high-pressure tube; The system was vacuumed and refilled with nitrogen three times, and then seal the tube; place the tube in an oil bath at a temperature of 80° C. to undertake a Suzuki coupling reaction for 8 hours, and then let the tube cool down to the ambient temperature; use diatomite to filter the resultant solution to remove metal; use ethyl acetate to extract the organic layer and remove water (the solvent); use a column chromatography device to purify the resultant; use a high temperature sublimation process to further purify the resultant to obtain the product of the synthesis reaction.

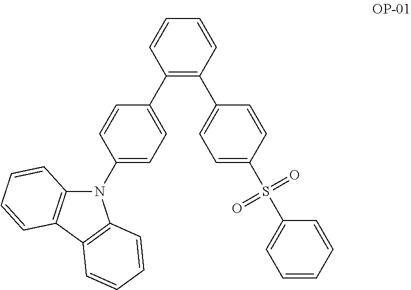 [SMILES: O=S(=O)(c1ccccc1)c1ccc(-c2ccccc2-c2ccc(-n3c4ccccc4c4ccccc43)cc2)cc1]

The Suzuki coupling reaction of 2-bromo-4′-(phenylsulfonyl)-1,1′-biphenyl (1.1 mmole) and (4-(9H-carbazol-9-yl)phenyl)boronic acid generates Product OP-01.

¹H NMR (400 MHz, CDCl₃), δ(ppm): 8.13(d, J=8.0, 2H) 7.91-7.89 (m, 2H) 7.87-7.84 (m, 2H) 7.56-7.53 (m, 1H) 7.52-7.49 (m, 1H) 7.47-7.44 (m, 2H) 7.43-7.39 (m, 5H) 7.38-7.36 (m, 1H) 7.34 (d, J=8.0, 4H) 7.30 (d, J=8.0, 3H) 7.27-7.26(m, 2H)

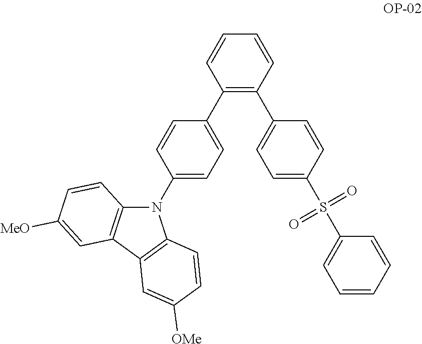 [SMILES: COc1ccc2c(c1)c1cc(OC)ccc1n2-c1ccc(-c2ccccc2-c2ccc(S(=O)(=O)c3ccccc3)cc2)cc1]

The Suzuki coupling reaction of 2-bromo-4′-(phenylsulfonyl)-1,1′-biphenyl (1.1 mmole) and (4-(3,6-dimethoxy-9H-carbazol-9-yl)phenyl)boronic acid generates Product OP-02.

¹H NMR (400 MHz, CDCl₃), δ(ppm): 7.91 (d, J=8.0, 2H) 7.85 (d, J=8.0, 2H) 7.55-7.53 (m, 3H) 7.51-7.48 (m, 1H) 7.47-7.45 (m, 2H) 7.44-7.40 (m, 3H) 7.34 (d, J=8.0, 2H) 7.26-7.22 (m, 4H) 7.06 (d, J=1.6, 1H) 7.04 (d, J=1.6, 1H) 3.95(s, 6H)

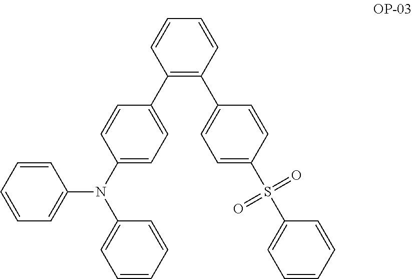 [SMILES: O=S(=O)(c1ccccc1)c1ccc(-c2ccccc2-c2ccc(N(c3ccccc3)c3ccccc3)cc2)cc1]

The Suzuki coupling reaction of 2-bromo-4′-(phenylsulfonyl)-1,1′-biphenyl (1.1 mmole) and (4-(diphenylamino)phenyl)boronic acid generates Product OP-03.

¹H NMR (400 MHz, CDCl₃), δ(ppm): 7.90-7.88 (m, 2H) 7.81-7.79 (m, 2H) 7.54-7.49 (m, 1H) 7.46-7.42 (m, 4H) 7.40-7.35 (m, 1H) 7.33-7.31 (m, 1H) 7.30-7.27 (m, 3H) 7.26-7.22 (m, 3H) 7.06-6.99 (m, 6H) 6.88-6.84 (m, 4H)

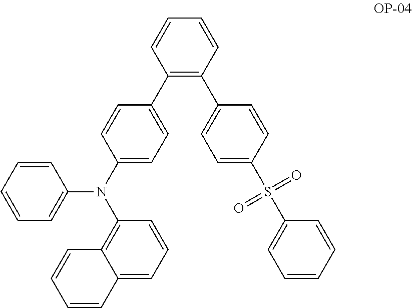 [SMILES: O=S(=O)(c1ccccc1)c1ccc(-c2ccccc2-c2ccc(N(c3ccccc3)c3cccc4ccccc34)cc2)cc1]

The Suzuki coupling reaction of 2-bromo-4′-(phenylsulfonyl)-1,1′-biphenyl (1.1 mmole) and (4-(naphthalen-1-yl(phenyl)amino)phenyl)boronic acid generates Product OP-04.

¹H NMR (400 MHz, CDCl₃), δ(ppm): 7.88-7.83 (m, 3H) 7.78-7.74 (m, 3H) 7.51-7.45 (m, 3H) 7.42-7.37 (m, 4H) 7.36-7.34 (m, 1H) 7.32-7.28 (m, 2H) 7.26-7.23 (m, 4H) 7.21-7.17 (m, 2H) 7.02-6.99 (m, 2H) 6.96-6.92 (m, 1H) 6.82-6.76 (m, 4H)

Refer to General Synthesis Formula (3) of the compound of the present invention:

General Synthesis Formula (3)

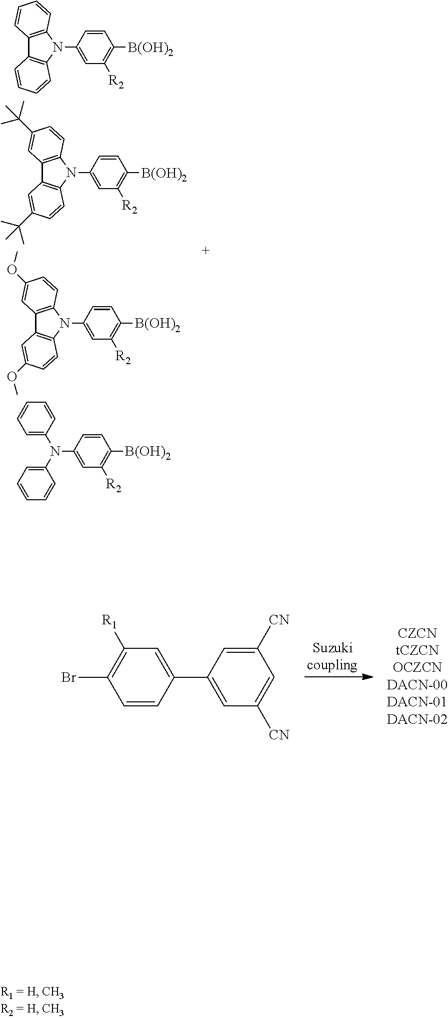

wherein group A in Representative Formula (3) is exemplified by the benzene dinitrile group; group D is exemplified by carbazole or diarylamine. It should be noted: the initial reactants having a given number of benzene rings will output an aromatic derivative having a corresponding number of benzene rings.

In the synthesis reaction, take a derivative of 4′-bromo-3′-methyl-[1,1′-biphenyl]-3,5-dicarbonitrile (1.1 mmole) and a boric acid derivative containing an amine group (1 mmole) into a high pressure tube; add toluene (3 mL), ethanol (1 mL), 2M potassium carbonate aqueous solution (1 mL), and Pd(PPh₃)₄ (0.1 mmole) into the high-pressure tube; The system was vacuumed and refilled with nitrogen three times, and then seal the tube; place the tube in an oil bath at a temperature of 80° C. to undertake a Suzuki coupling reaction for 24 hours, and then let the tube cool down to the ambient temperature; use diatomite to filter the resultant solution to remove metal; use ethyl acetate to extract the organic layer and remove water (the solvent); use a column chromatography device to purify the resultant; use a high temperature sublimation process to further purify the resultant to obtain the product of the synthesis reaction.

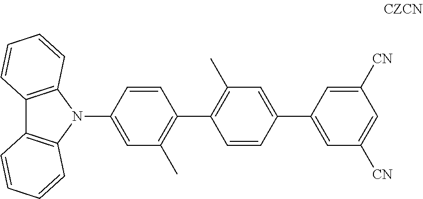 [SMILES: Cc1cc(-c2cc(C#N)cc(C#N)c2)ccc1-c1ccc(-n2c3ccccc3c3ccccc32)cc1C]

The Suzuki coupling reaction of (4-(9H-carbazol-9-yl)-2-methylphenyl)boronic acid and 4-bromo-3-methyl-[1,1-biphenyl]-3,5-dicarbonitrile generates Product CZCN.

¹H NMR (400 MHz, CDCl₃), δ(ppm): 0.16-8.14(m,4H)7.90(s,1H)7.51-7.27(m,12H)2.27(s,3H)2.18(s,3H)

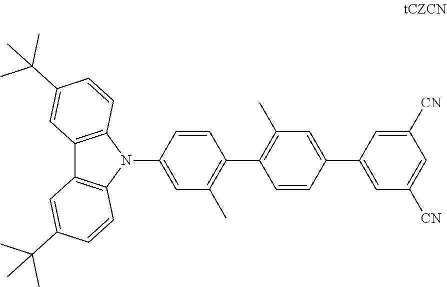 [SMILES: Cc1cc(-c2cc(C#N)cc(C#N)c2)ccc1-c1ccc(-n2c3ccc(C(C)(C)C)cc3c3cc(C(C)(C)C)ccc32)cc1C]

The Suzuki coupling reaction of (4-(3,6-di-tert-butyl-9H-carbazol-9-yl)-2-methylphenyl)boronic acid and 4-bromo-3-methyl-[1,1-biphenyl]-3,5-dicarbonitrile generates Product tCZCN.

¹H NMR (400 MHz, CDCl₃), δ(ppm):8.14(s,4H)7.9(s,1H) 7.5-7.42(m,8H)7.39(d,J=8,1H)7.29(d,J=8.4,1 H)2.26(s,3H)2.16(s,3H)

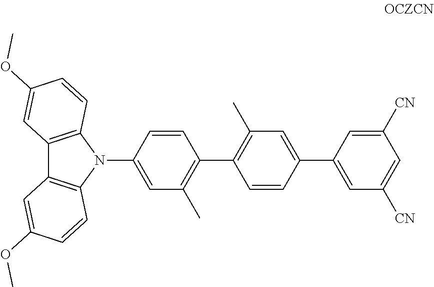 [SMILES: COc1ccc2c(c1)c1cc(OC)ccc1n2-c1ccc(-c2ccc(-c3cc(C#N)cc(C#N)c3)cc2C)c(C)c1]

The Suzuki coupling reaction of (4-(3,6-dimethoxy-9H-carbazol-9-yl)-2-methylphenyl)boronic acid and 4-bromo-3-methyl-[1,1-biphenyl]-3,5-dicarbonitrile generates Product OCZCN.

¹H NMR (400 MHz, CDCl₃), δ(ppm): 8.14(s,2H)7.90(t,J=1.6,1H)7.56(d,J=2.8,2H)7.50-7.40(m,6H)7.35(d,J=8,1H)7.29(d,J=8,1H)7.06(d,J=2.8,1H)7.04(d,J=2.4,1 H)3.95(s,6H)2.25(s,3H)2.16(s, 3H)

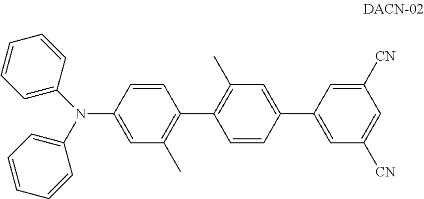 [SMILES: Cc1cc(-c2cc(C#N)cc(C#N)c2)ccc1-c1ccc(N(c2ccccc2)c2ccccc2)cc1C]

The Suzuki coupling reaction of 4-(diphenylamino)-2-methylphenyl)boronic acid and 4-bromo-3-methyl-[1,1-biphenyl]-3,5-dicarbonitrile generates Product DACN-02.

¹H NMR (400 MHz, CDCl₃), δ(ppm): 8.10(s,2H)7.87(s,1H)7.43 (s,1H)7.39(d,J=8,1H)7.29-7.26(m,4H)7.12(d,J=7.6, 4H)7.03-6.91(m,6H)2.19(s,3H)1.96(s,3H)

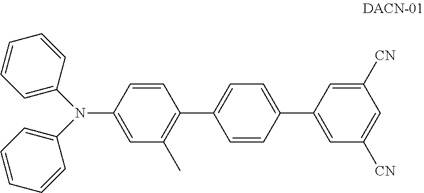 [SMILES: Cc1cc(N(c2ccccc2)c2ccccc2)ccc1-c1ccc(-c2cc(C#N)cc(C#N)c2)cc1]

The Suzuki coupling reaction of (4-(diphenylamino)-2-methylphenyl)boronic acid and 4-bromo-[1,1-biphenyl]-3,5-dicarbonitrile generates Product DACN-01.

¹H NMR (400 MHz, CDCl₃), δ(ppm):8.11(s,2H)7.88(s,1H)7.58(d,J=8.4,2H)7.48(d,J=8,2H)7.28-7.26(m,3H) 7.14-7.10(m,5H)7.04-6.95(m,5H)2.2(s,3H)

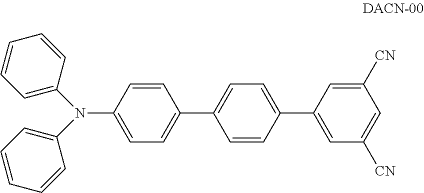 [SMILES: N#Cc1cc(C#N)cc(-c2ccc(-c3ccc(N(c4ccccc4)c4ccccc4)cc3)cc2)c1]

The Suzuki coupling reaction of (4-(diphenylamino)phenyl)boronic acid and 4-bromo-[1,1-biphenyl]-3,5-dicarbonitrile generates Product DACN-00.

¹H NMR (400 MHz, CDCl₃), δ(ppm): 8.10(s,2H)7.87(s,1H)7.70(d, J=8,2H)7.59(d, J=8.4,2H)7.50(d, J=8.4,2H)7.29-7.25(m,4H)7.15-7.12(m,6H)7.05(t, J=7.2,2H)

TABLE.1
the absorption spectra and emission spectra of the compounds
of the present invention

| | In Toluene | In Toluene |
|---|---|---|
| Compound | λ_{abs} (nm) | λₑₘ (nm) |
| BP-01 | 290, 328, 340 | 396 |
| BT-01 | 292, 325, 340 | 348, 364 |
| BT-02 | 285, 328, 342 | 351, 369 |
| BT-03 | 311, 354. 374 | 388 |
| BT-04 | 304 | 407 |
| BT-05 | 292 | 409 |
| OP-01 | 291, 326, 341 | 409 |
| OP-02 | 312, 354, 371 | 443 |
| OP-03 | 305 | 449 |
| OP-04 | 310 | 442 |
| CzCN | 291, 340 | 384 |
| tCzCN | 298, 332, 346 | 381 |
| OCzCN | 310, 354, 372 | 388, 407 |
| DACN-02 | 299 | 433 |
| DACN-01 | 294, 341 | 439 |
| DACN-00 | 292, 362 | 444 |

Refer to the sole FIGURE, a diagram schematically showing the structure of a light emitting element using aromatic derivatives according to one embodiment of the present invention. The light emitting element comprises an anode 1, a cathode 2 and a light emitting layer 3 containing compounds. In the light emitting layer 3, the host material is doped with a light emitting material. The structure of the light emitting element further comprises a hole injection layer 7, a hole transport layer 4, an electron blocking layer 9, an light emitting layer 3, an exciton blocking layer 10, a hole blocking layer 6, an electron transport layer 5, and an electron injection layer 8, which are arranged bottom up in sequence from the anode 1. The sole FIGURE does not depict the real thicknesses of the layers but only schematically demonstrates the structure of the light emitting element. The real thicknesses of the layers are irrespective of the dimensions shown in the sole FIGURE. In the present invention, the hole injection layer 7, the electron blocking layer 9, the exciton blocking layer 10, the hole blocking layer 6 and the electron injection layer 8 are optional components. The aromatic compounds of the present invention may be dopant materials or host materials of the light emitting layer. Besides, the aromatic compounds of the present invention may be electron transport materials or hole transport materials.

The structure of the light emitting element uses an ITO (Indium Tin Oxide) substrate and electrodes containing LiF/Al; the tested dopant material is Fir(Pic); the tested electron transport layer includes TAZ, BCP, TmpyPb, and TPBI, which can be used in the hole blocking layer or used in the hole blocking layer and electron transport layer simultaneously; the tested hole transport layer includes NPB (4,4′-bis[N-(1-naphthyl)-N-phenyl-amino]bipheny) and MCP, which can be used in the electron blocking layer or used in the electron blocking layer and hole transport layer simultaneously.

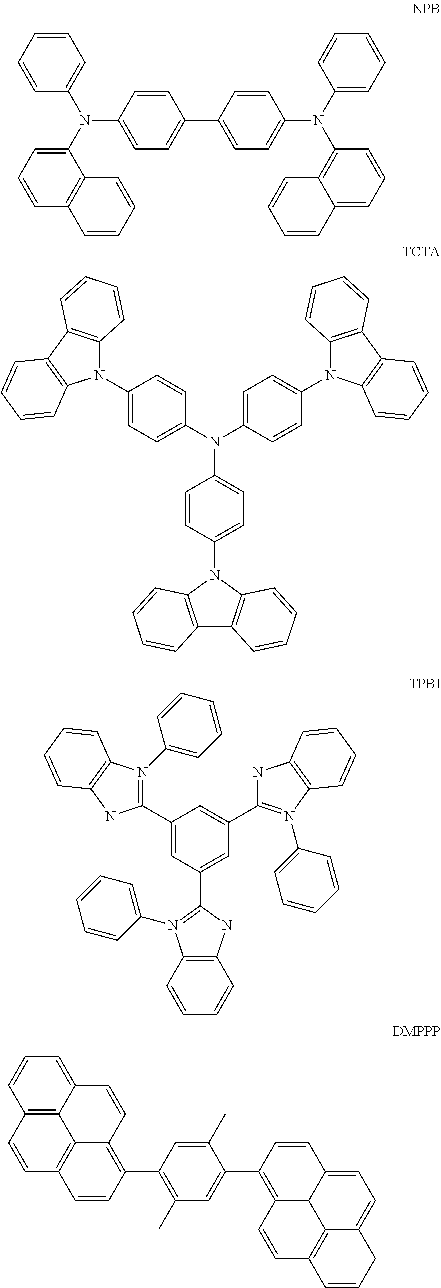

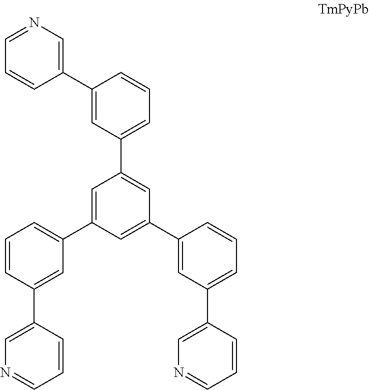 [SMILES: c1cncc(-c2cccc(-c3cc(-c4cccc(-c5cccnc5)c4)cc(-c4cccc(-c5cccnc5)c4)c3)c2)c1]

The elements are briefly described below according to one embodiment of the present invention.

A: NPB(30)/MCP (20)/BT-01:Fir(Pic)(8%)(30)/TAZ(50)/LiF(1)/Al(100)

B: NPB(30)/MCP (20)/BT-01:Fir(Pic)(8%) (30)/TAZ(40))/LiF(1)/Al(100)

C: NPB(30)/MCP (20)/BT-01:Fir(Pic)(8%) (30)/TAZ(30)/LiF(1)/Al(100)

D: NPB(30)/MCP (20)/BT-01:Fir(Pic)(8%) (30)/TPBI(40)/LiF(1)/Al(100)

E: NPB(30)/MCP (20)/BT-01:Fir(Pic)(8%) (30)/BCP(40)/LiF(1)/Al(100)

F: NPB(30)/MCP(20)/BT-01:Fir(Pic)(8%)(30)/B3PYmpm(40)/LiF(1)/Al(100)

G: NPB(30)/MCP (20)/BT-01:Fir(Pic)(8%) (30)/TmpyPb(40)/LiF(1)/Al(100)

H: NPB(30)/MCP (20)/BT-01:Fir(Pic)(8%) (30)/TmpyPb(30)/LiF(1)/Al(100)

I: NPB(30)/MCP (20)/BT-01:Fir(Pic)(8%) (30)/TmpyPb(50)/LiF(1)/Al(100)

J: NPB(30)/MCP (20)/BT-01:Fir(Pic)(8%) (30)/TmpyPb(60)/LiF(1)/Al(100)

K: NPB(30)/MCP (20)/BT-01:Fir(Pic)(8%) (30)/TmpyPb(70)/LiF(1)/Al(100)

TABLE 2
the performance of the elements using the compounds of the present invention

| | | | | Lₘₐₓ | | EL | |
|---|---|---|---|---|---|---|---|
| | E.Q.E. | C.E. | P.E. | (cd/m²) | V_{d} | λₘₐₓ | |
| Element | (%) (V) | (cd/A) | (lm/W) | (V) | (V) | (nm) | CIE_{(x, y)} |
| A | 24.3(5.5) | 58.3 | 35.5 | 43525(14.5) | 4.2 | 474 | (0.16, 0.38) |
| B | 23.5(5.5) | 52.2 | 36.9 | 35584(12.0) | 3.7 | 474 | (0.14, 0.35) |
| C | 19.8(6.0) | 42.1 | 29.5 | 33855(12.5) | 3.7 | 474 | (0.14, 0.32) |
| D | 24.8(5.5) | 47.7 | 33.4 | 38699(12.5) | 3.3 | 472 | (0.13, 0.32) |
| E | 20.1(5.5) | 41.1 | 27.6 | 33434(14.5) | 3.7 | 472 | (0.13, 0.32) |
| F | 19.4(5.0) | 42.5 | 33.1 | 36619(12.5) | 3.3 | 474 | (0.14, 0.35) |
| G | 28.1(4.5) | 64.7 | 54.7 | 41201(12.0) | 3.3 | 474 | (0.14, 0.37) |
| H | 23.5(5.5) | 47.7 | 33.4 | 38699(11.5) | 3.2 | 472 | (0.14, 0.31) |
| I | 29.4(4.5) | 63.5 | 54.6 | 46673(12.0) | 3.2 | 474 | (0.14, 0.35) |
| J | 29.0(5.0) | 66.2 | 53.5 | 47567(12.5) | 3.2 | 474 | (0.15, 0.37) |
| K | 27.2(5.0) | 64.7 | 53.6 | 47749(12.5) | 3.3 | 474 | (0.15, 0.37) |

Lₘₐₓ: maximum luminescence;
E.Q.E.: maximum external quantum efficiency;
C.E.: maximum current efficiency;
P.E.: maximum power efficiency);
V_{d}: Drive voltage);
λₘₐₓ: maximum emission wavelength

As shown in Table.2, the maximum external quantum efficiencies of Elements G, I, J and K, which use the compounds of the present invention, exceed 27 and respectively reach 28.1, 29.4, 29.0 and 27.2.

The elements are briefly described below according to another embodiment of the present invention.

L: NPB(30)/MCP (20)/BT-01:2CzPN(8%) (30)/TmpyPb(50)/LiF(1)/Al(100)

M: NPB(30)/MCP (20)/BT-03:2CzPN(8%) (30)/TmpyPb(50)/LiF(1)/Al(100)

N: NPB(30)/MCP (20)/BT-04:2CzPN(8%) (30)/TmpyPb(50)/LiF(1)/Al(100)

O: NPB(30)/MCP (20)/BT-05:2CzPN(8%) (30)/TmpyPb(50)/LiF(1)/Al(100)

TABLE 3
the performance of the elements using the compounds of the present invention

| | | | | Lₘₐₓ | | EL | |
|---|---|---|---|---|---|---|---|
| | E.Q.E. | C.E. | P.E. | (cd/m²) | V_{d} | λₘₐₓ | |
| Element | (%) (V) | (cd/A) | (lm/W) | (V) | (V) | (nm) | CIE_{(x, y)} |
| L | 25.4(3.5) | 49.8 | 44.6 | 13974(15.0) | 3.2 | 482 | (0.15, 0.29) |
| M | 2.8(6.0) | 9.1 | 5.9 | 10118(15.0) | 3.6 | 530 | (0.32, 0.53) |
| N | 18.4(4.0) | 55.4 | 47.2 | 12490(13.0) | 3.4 | 522 | (0.27, 0.49) |
| O | 2.4(6.5) | 5.6 | 4.1 | 9209(15.5) | 4.0 | 494 | (0.20, 0.36) |

The elements are briefly described below according to further another embodiment of the present invention.

P: NPB(30)/MCP (20)/BT-01:8 wt % tCzCN(30)/TmpyPb(50)/LiF(1)/Al(100)

Q: NPB(30)/MCP (20)/BT-01:8 wt % tDACN-02(30)/TmpyPb(50)/LiF(1)/Al(100)

R: NPB(10)/TCTA (40)/DMPPP:5 wt % tDACN-01(30)/TPBi(30)/LiF(1)/Al(100)

S: NPB(10)/TCTA (40)/DMPPP:5 wt % tDACN-00(30)/TPBi(30)/LiF(1)/Al(100)

TABLE 4
the performance of the elements using the compounds of the present invention

| | | Max | Max | | | | |
|---|---|---|---|---|---|---|---|
| | | C.E. | P.E. | Lₘₐₓ | | EL | |
| | E.Q.E. | (cd/A), | (lm/W), | (cd/m²) | V_{d} | λₘₐₓ | |
| Element | (%) (V) | V | V | (V) | (V) | (nm) | CIE_{(x, y)} |
| P | 1.9(6.5) | 0.9, 6.5 | 0.5, 5,5 | 853, 12.0 | 4.2 | 412 | (0.15, 0.08) |
| Q | 3.9(6.0) | 3.5, 6.0 | 1.9, 6.0 | 3887, 13.0 | 4.2 | 452 | (0.14, 0.10) |
| R | 6.0(9.5) | 4.9, 9.5 | 1.9, 7.0 | 20450, 14.5 | 5.3 | 442 | (0.15, 0.09) |
| S | 6.5(10.0) | 5.6, 10.0 | 2.1, 7.5 | 26803, 15.0 | 5.5 | 445 | (0.15, 0.09) |

In conclusion, the present invention proposes an aromatic derivative whose two ends respectively have an electron donating group and an electron accepting group. The aromatic derivative of the present invention can emit blue or green light and function as a host or dopant light emitting material. The OLED using the aromatic derivatives of the present invention has superior performance.

What is claimed is:

1. An aromatic compound expressed by Formula (I): 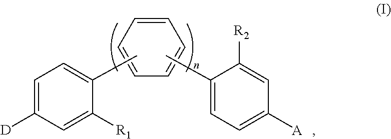
wherein n is an integer ranging from 0-3; each of R₁ and R₂ is independently a hydrogen atom, a C1-C6 alkyl group, or a phenyl group; A is a substituted or unsubstituted benzenesulfonic acid, or a substituted or unsubstituted benzene dinitrile group; D is a substituted or unsubstituted diarylamine group, or a substituted or unsubstituted nitrogen-containing heteroaryl group.

2. The aromatic compound according to claim 1, wherein said group D is bonded to a benzene ring through a nitrogen atom.

3. The aromatic compound according to claim 1, wherein said nitrogen-containing heteroaryl group is a heteroaryl group with two benzene rings fused with a central heteroaryl group.

4. The aromatic compound according to claim 1, wherein said nitrogen-containing heteroaryl group is carbazole, 4aH-phenoxazine, or acridine.

5. The aromatic compound according to claim 1, wherein said benzene dinitrile group is 3, 5-benzene dinitrile.

6. The aromatic compound according to claim 1, wherein said n is 0 or 1.

7. The aromatic compound according to claim 1, wherein each of said R₁ and said R₂ is independently a hydrogen atom or a methyl group.

8. An organic light emitting diode comprising
a cathode;
an anode; and
a light emitting layer arranged between said cathode and said anode and including a light emitting material, wherein said light emitting material is an aromatic compound expressed by Formula (I): 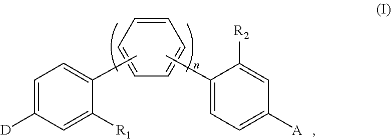
wherein n is an integer ranging from 0-3; each of R₁ and R₂ is independently a hydrogen atom, a C1-C6 alkyl group, or a phenyl group; A is a substituted or unsubstituted benzenesulfonic acid, or a substituted or unsubstituted benzene dinitrile group; and D is a substituted or unsubstituted diarylamine group, or a substituted or unsubstituted nitrogen-containing heteroaryl group.

9. The aromatic compound according to claim 8, wherein said group D is bonded to a benzene ring through a nitrogen atom.

10. The aromatic compound according to claim 8, wherein said nitrogen-containing heteroaryl group is a heteroaryl group with two benzene rings fused with a central heteroaryl group.

11. The aromatic compound according to claim 8, wherein said nitrogen-containing heteroaryl group is carbazole, 4aH-phenoxazine, or acridine.

12. The aromatic compound according to claim 8, wherein said benzene dinitrile group is 3, 5-benzene dinitrile.

13. The aromatic compound according to claim 8, wherein said n is 0 or 1.

14. The aromatic compound according to claim 8, wherein each of said R₁ and said R₂ is independently a hydrogen atom or a methyl group.

15. The organic light emitting diode according to claim 8, wherein said light emitting material is a host material or a dopant material.

16. The organic light emitting diode according to claim 8, wherein the organic light emitting diode is a blue light organic light emitting diode or a green light organic light emitting diode.
